(19) United States
(12) Patent Application Publication
(10) Pub. No.: US 2014/0314288 A1
(43) Pub. Date: Oct. 23, 2014

(54) Method and apparatus to detect lesions of diabetic retinopathy in fundus images

(71) Applicant: Keshab K. Parhi, Maple Grove, MN (US)

(72) Inventors: Sohini Roychowdhury, Minneapolis, MN (US); Keshab K. Parhi, Maple Grove, MN (US); Dara D. Koozekanani, Minneapolis, MN (US)

(21) Appl. No.: 14/120,027

(22) Filed: Apr. 16, 2014

Related U.S. Application Data
(60) Provisional application No. 61/854,034, filed on Apr. 17, 2013.

(51) Int. Cl. G06T 7/00; A61B 3/12

(52) U.S. Cl. CPC G06T 7/0012; A61B 3/1241
USPC 382/128

(57) ABSTRACT

The present invention relates to the design and implementation of a three stage computer-aided screening system that analyzes fundus images with varying illumination and fields of view, and generates a severity grade for diabetic retinopathy (DR) using machine learning. In the first stage, bright and red regions are extracted from the fundus image. An optic disc has similar structural appearance as bright lesions, and the blood vessel regions have similar pixel intensity properties as the red lesions. Hence, the region corresponding to the optic disc is removed from the bright regions and the regions corresponding to the blood vessels are removed from the red regions. This leads to an image containing bright candidate regions and another image containing red candidate regions. In the second stage, the bright and red candidate regions are subjected to two-step hierarchical classification. In the first step, bright and red lesion regions are separated from non-lesion regions. In the second step, the classified bright lesion regions are further classified as hard exudates or cotton-wool spots, while the classified red lesion regions are further classified as hemorrhages and micro-aneurysms. In the third stage, the numbers of bright and red lesions per image are combined to generate a DR severity grade. Such a system will help in reducing the number of patients requiring manual assessment, and will be critical in prioritizing eye-care delivery measures for patients with highest DR severity.

### CROSS-REFERENCE TO RELATED APPLICATIONS

This application claims the benefit of U.S. Provisional Application No. 61/854,034, filed on Apr. 17, 2013, the entire content of which is incorporated herein by reference in its entirety.

### FIELD OF THE INVENTION

Automated detection of diabetic retinopathy (DR) lesions from fundus images is important for detecting ophthalmic abnormalities and for developing cost-effective DR screening systems that will help in grading severity of non-proliferative DR. This will enhance the effectiveness of the present day eye-care delivery.

### BACKGROUND OF THE INVENTION

According to a study by the American Diabetes Association, diabetic retinopathy (DR) had affected more than 4.4 million Americans of age 40 and older during 2005-2008, with almost 0.7 million (4.4% of those with diabetes) having advanced DR that could lead to severe vision loss. Early detection and treatment of DR can provably decrease the risk of severe vision loss by over 90%. Thus, there is a high consensus for the need of efficient and cost-effective DR screening systems.

Unfortunately almost 50% of diabetic patients in the United States currently do not undergo any form of documented screening exams in spite of the guidelines established by the American Diabetes Association (ADA) and the American Academy of Ophthalmology (AAO). Statistics show that 60% of the patients requiring laser surgery to prevent blindness do not receive treatment. The major reasons for this screening and treatment gap include insufficient referrals, economic hindrances and insufficient access to proper eye care. Telemedicine, with distributed remote retinal fundus imaging and grading at either local primary care offices or centralized grading remotely by eye care specialists, has increased access to screening and follow-up necessary treatment.

Computer-aided screening systems have recently gained importance for increasing the feasibility of DR screening, and several algorithms have been developed for automated detection of lesions such as exudates, hemorrhages and micro-aneurysms. So far an automated DR screening system, Medalytix (See, G. S. Scotland, P. McNamee, A. D. Fleming, K. A. Goatman, S. Philip, G. J. Prescott, P. F. Sharp, G. J. Williams, W. Wykes, G. P. Leese, and J. A. Olson, “Costs and consequences of automated algorithms versus manual grading for the detection of referable diabetic retinopathy,” British Journal of Ophthalmology, vol. 94, no. 6, pp. 712-719, 2010), has been used for screening normal patients without DR from abnormal patients with DR on a local data set, with sensitivity in the range 97.4-99.3% on diabetic patients in Scotland. The screening outcome combined with manual analysis of the images that are classified as abnormal by the automated system has shown to reduce the clinical workload by more than 25% in Scotland. Another automated DR screening system grades images from a local data set for unacceptable quality, referable, non-referable DR with sensitivity 84% and specificity 64% (See, M. D. Abramoff, M. Niemeijer, M. S. Suttorp-Schulten, M. A. Viergever, S. R. Russell, and B. van Ginneken, “Evaluation of a system for automatic detection of diabetic retinopathy from color fundus photographs in a large population of patients with diabetes,” Diabetes Care, vol. 31, no. 2, pp. 193-198, February 2008). Both these automated systems motivate the need for a fast and more accurate DR screening and prioritization system such as the proposed invention.

### BRIEF SUMMARY OF THE INVENTION

Details of the algorithms and apparatus for automated detection of diabetic retinopathy lesions in fundus images are provided. As described herein, the present invention can be used for screening patients with mild, moderate to severe non-proliferative DR, and to prioritize follow-up treatment based on the DR severity.

One aspect of the proposed invention is the 3-stage system design where each stage has minimal run-time complexity to ensure a fast DR detection system. An optimal feature set is defined that will allow classifiers to detect retinopathy lesions and to generate a severity grade for a fundus image (See, S. Roychowdhury, D. Koozekanani, and K. K. Parhi, “DREAM: Diabetic Retinopathy Analysis using Machine Learning,” IEEE Journal of Biomedical and Health Informatics, 2014, doi: 10.1109JBHI.2013.2294635).

A key contribution of the proposed invention is a novel two-step hierarchical binary classification method that rejects false positives in the first step and in the second step, bright lesions are classified as cotton wool spots (CWS) or hard exudates (HE), and red lesions are classified as hemorrhages (HA) and micro-aneurysms (MA), respectively. This hierarchical classification method reduces the time complexity by 18-24% over a parallel classification method that trains separate classifiers for identifying CWS, HE, HA and MA from false positives.

In an embodiment, the green plane of the color fundus image is pre-processed by a high pass filter and subsequently thresholded to extract bright candidate regions and red candidate regions. Other embodiments for extracting the bright and red candidate regions can also be used.

In an embodiment, using region-based features, the red and bright candidate regions are classified using a k-Nearest Neighbor (kNN) and Gaussian Mixture Model (GMM) classifier, respectively. In other embodiments, other classifiers may be used for lesion classification.

In an embodiment, the number and type of red lesions detected per image are combined using the Early Treatment Diabetic Retinopathy Study (ETDRS) scale to generate a DR severity grade. In another embodiment, the number and type of bright and red lesions detected per image are combined using the International Clinical Diabetic Retinopathy Disease Severity (ICDRS) scale for the DR severity grade. In other embodiments different criteria for choice of bright and/or bright lesions may be used for determining the DR severity grade.

Further embodiments, features, and advantages of the present invention, as well as the structure and operation of the various embodiments of the present invention are described in detail below with reference to accompanying figures.

### BRIEF DESCRIPTION OF THE DRAWINGS/FIGURES

The present invention is described with reference to the accompanying figures. The accompanying figures, which are incorporated herein, form part of the specification, illustrate the present invention, and, together with the description, further serve to explain the principles of the invention and to enable a person skilled in the relevant art to make and use the invention.

FIG. 1 illustrates the flow diagram for extraction of bright candidate regions from a fundus image;

FIG. 2 illustrates the flow diagram for extraction of red candidate regions from a fundus image;

FIG. 3 is a flow diagram illustrating the two-step hierarchical classification method for detecting bright lesions such as hard exudates, and cotton-wool spots among the bright candidate regions;

FIG. 4 is a flow diagram illustrating the two-step hierarchical classification method for detecting red. lesions such as hemorrhages, and micro-aneurysms among the red candidate regions;

FIG. 5 illustrates the flow diagram for combining the number of red lesions to generate a DR severity grade following the ETDRS scale;

FIG. 6 illustrates the flow diagram for combining the number of bright and red lesions to generate a DR severity grade following the ICDRS scale;

FIG. 7. illustrates an example of bright candidate region extraction;

FIG. 8 illustrates an example of red candidate region extraction;

FIG. 9 illustrates an example of two-step hierarchical bright lesion classification;

FIG. 10 illustrates an example of two-step hierarchical red lesion classification;

FIG. 11 lists the 30 features used for classification of the right and red candidate regions;

FIG. 12 shows the performance of the proposed system on 1200 images from the MESSIDOR data set;

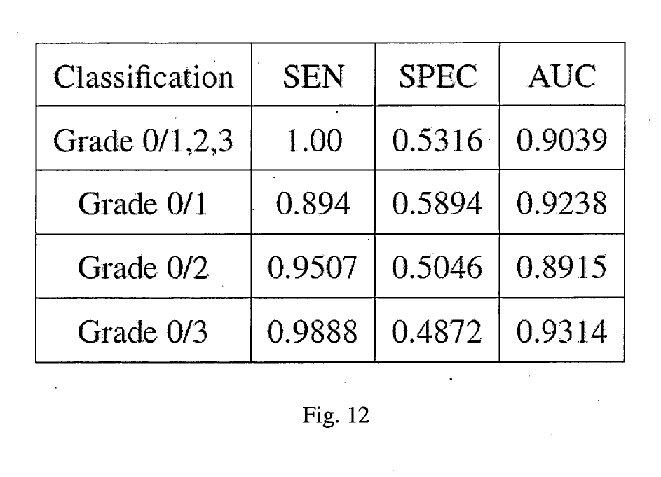

FIG. 13 illustrates a block diagram of an exemplary web-based system that can utilize the disclosed DR lesion detection and grading invention;

FIG. 14 illustrates a block diagram of an exemplary stand-alone device for detecting diabetic retinopathy lesions and severity grade;

FIG. 15 illustrates a block diagram of an exemplary fundus camera integrated with an apparatus that utilizes the disclosed lesion detection system.

### DETAILED DESCRIPTION OF THE INVENTION

Proposed Invention

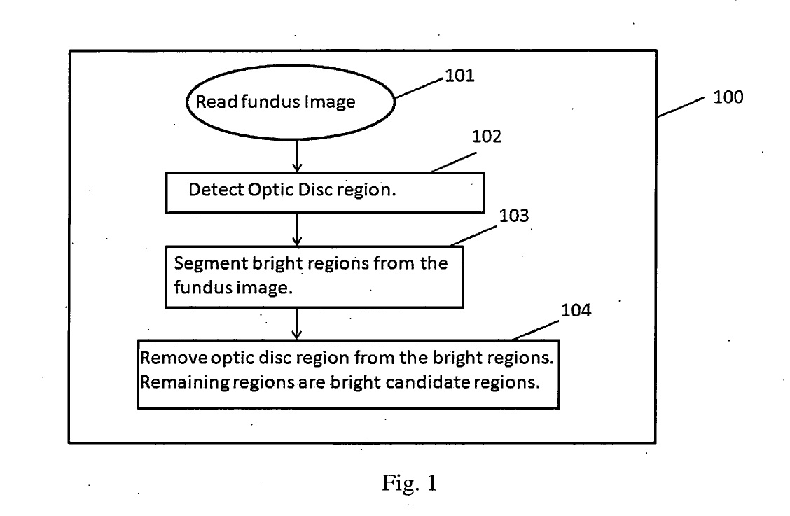

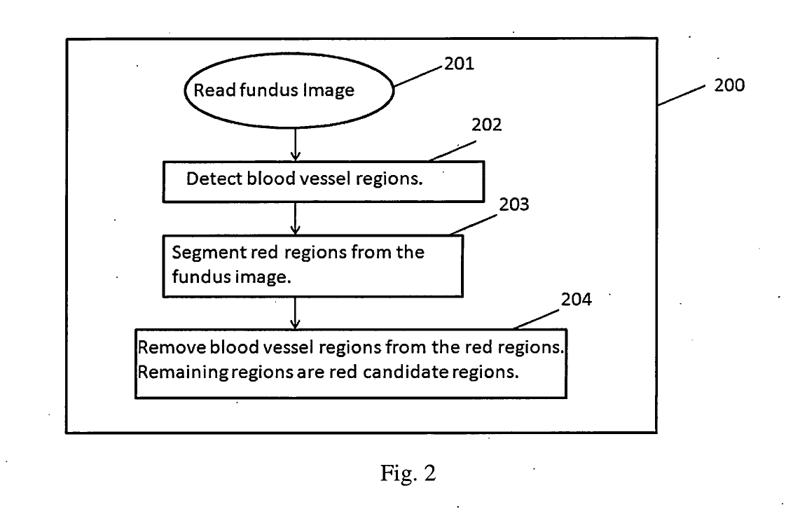

The disclosed invention comprises of a 3-stage algorithm to automatically detect and grade the severity of DR using retinal fundus images. In the first stage, bright regions and red regions are detected from the fundus image. In one embodiment, the green plane of a fundus image is subjected to high-pass filtering and thresholding to detect regions that are brighter or darker than their immediate neighborhood regions. These regions correspond to bright candidate regions and red candidate regions, respectively. Since the optic disc (OD) region has similar appearance as the bright lesions, and the blood vessel regions have similar pixel intensities as the red lesions, hence it is imperative to detect the OD region and blood vessel regions early on and mask out those regions to prevent false detections of retinopathy lesions. The steps for identifying the bright candidate regions and red candidate regions are shown in FIG. 1 and FIG. 2, respectively.

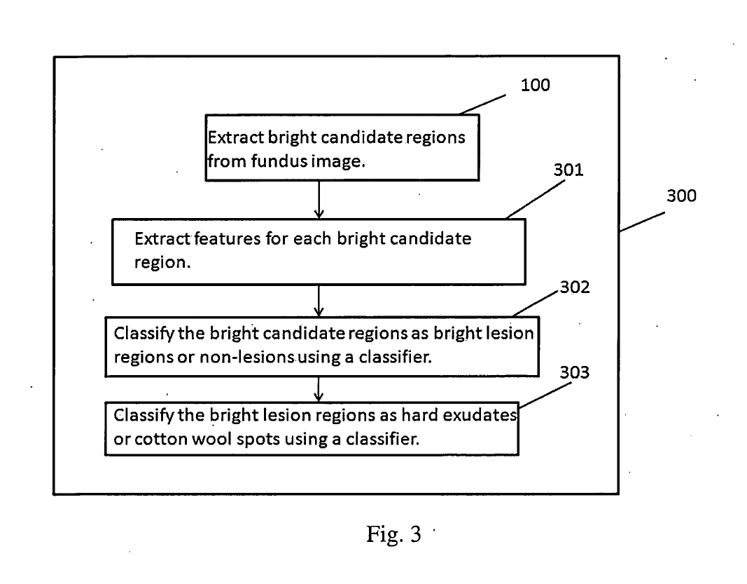

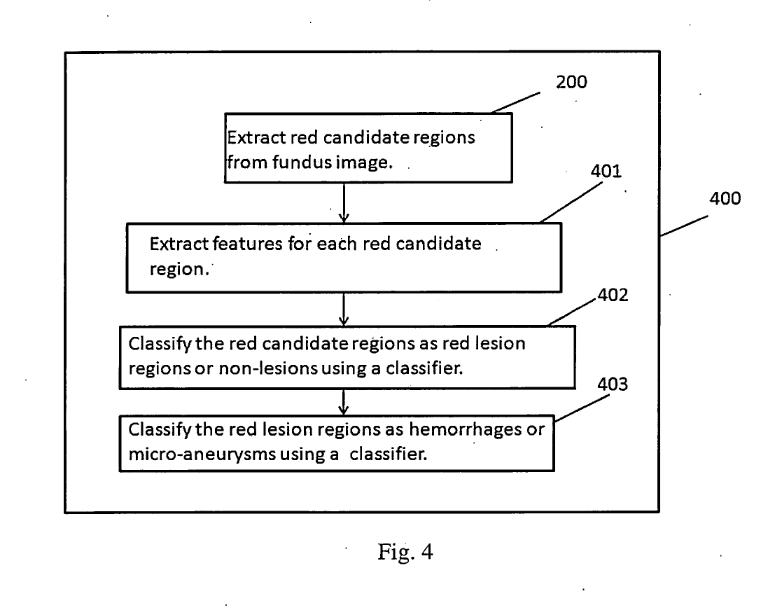

In the second stage, the bright candidate regions and the red candidate regions are subjected to feature-based classification. Corresponding to each candidate region, region and pixel based features are extracted. In one embodiment, 30 discriminating features are extracted for each region. Other combinations of features may also be used in other embodiments. Next, each bright or red candidate region is classified in two hierarchical steps. In the first step, the bright/red candidate regions are classified as bright/red lesion regions or non-lesion regions, such that, non-lesions (or false positive regions) are eliminated from the candidate regions. In the second step, the bright lesion regions are further classified as hard exudates or cotton-wool spots, and the red lesion regions are further classified as hemorrhages or micro-aneurysms. These lesion classification steps for bright and red lesions are shown in FIG. 3 and FIG. 4, respectively.

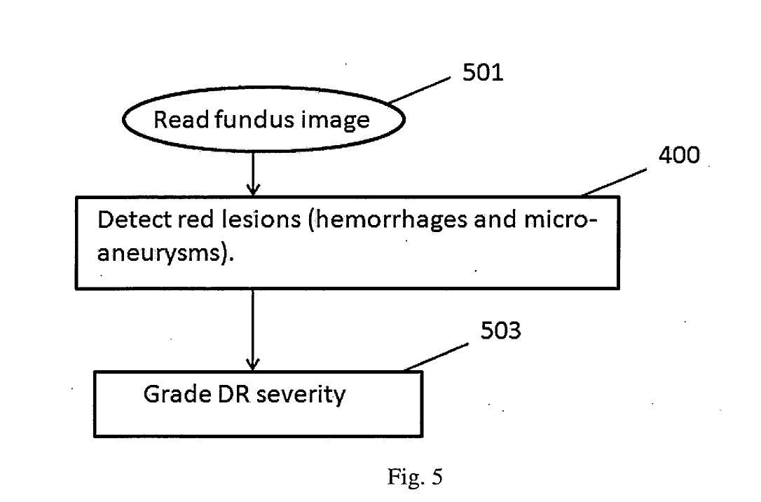

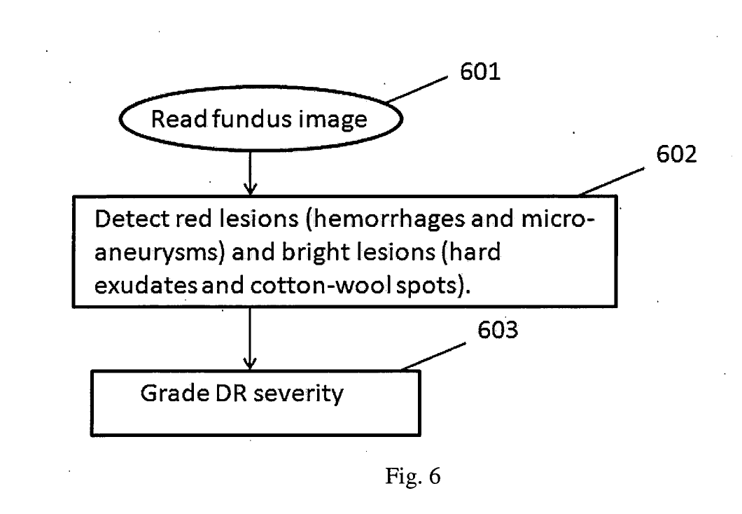

In the third stage, the numbers of lesions detected per image are combined using well-known lesion combination scales to generate a DR severity grade. While a DR grade 0 refers to a normal patient with no DR, grades 1, 2, 3 refer to increasing severities of DR, i.e., mild, moderate and severe DR, respectively. In one embodiment, the ETDRS scale can be used to generate the DR severity grade as shown in FIG. 5, while in another embodiment, the ICDRS scale may be used as shown in FIG. 6. In other embodiments, other grading mechanisms may be used.

Extraction of Candidate Regions

The steps for extracting bright candidate regions are shown in FIG. 1 block 100. In block 101, the fundus image is received. In one embodiment, the green plane of the fundus image is pre-processed by histogram equalization and contrast enhancement, followed by scaling all pixel intensities in the range [0,1] resulting in image I. The OD region might become a false positive for bright lesion detection if it is not removed at an early stage of the automated detection algorithm. Hence, an algorithm for automated detection of the OD neighborhood region is invoked in block 102. Various embodiments could make use of different OD detection algorithms.

To segment the bright regions in the image, in one embodiment of block 103, I is morphologically eroded using a linear structuring element of length 50 pixels and width 1 pixel, followed by image reconstruction. In other embodiments other structures of structuring element may be used. In one embodiment, the reconstructed image is subtracted from I, and normalized and subjected to contrast enhancement to yield image I_{b}. Next, I_{b} is normalized and globally thresholded using Otsu's threshold to segment the bright regions in image I_{BR}. In other embodiments, other thresholds may be used. Finally, the OD region is removed from the bright regions in I_{BR} in block 104, resulting in an image containing bright candidate regions R_{BR}. Various other embodiments may extract the bright candidate regions using different approaches.

The steps for extracting red candidate regions are shown in FIG. 2 block 200. The fundus image is received in block 201, followed by the detection of blood vessel regions in block 202. Blood vessel regions need to be detected in early stages of the automated lesion detection algorithm to reduces instances of false positives in later stages. In one embodiment, a low-pass filtered version of the green plane image I is estimated by median filtering I. Next, a high-pass filtered version of the green plane image is obtained by subtracting the low-pass filtered image from the original image. From this high-pass filtered image, only the negative pixel values are retained while positive pixel values are ignored. This negative thresholded high-pass filtered image contains the red regions. The absolute value of the pixels in the red region image are resealed in [0,1] range and region-grown to detect the major blood vessel regions.

In block 203, the red regions from the, thresholded high-pass filtered image are detected. In block 204, the blood vessel regions are removed from the red regions and the remaining regions are the red candidate regions R_{RR}. Other embodiments may extract the red candidate regions using different approaches.

Lesion Classification

Following the detection of bright and red candidate regions, each candidate region is subjected to classification for two reasons. The first reason is that feature-based classification helps to eliminate false positive regions. The second reason is that classification helps to distinguish between the different kinds of lesions. For instance, in FIG. 3 block 300, bright candidate regions (R_{BR}) are received from block 100. Region and pixel-based features are computed for each candidate region in block 301. Examples of region-based features include area, perimeter, solidity of a particular region. Examples of pixel-based features include minimum, maximum, mean or standard deviation of pixel intensity values within a region. Next, each bright candidate region is classified as bright lesion regions (R_{BL}) and non-lesion regions (R_{NBL}) in block 302. Non-lesion regions R_{NBL} represent false-positives and are not considered any further. All the regions that were classified as bright lesions (R_{BL}) are further classified as hard exudate regions (R_{HE}) or cotton-wool spot regions (R_{CWS}) in block 303.

Similarly, in FIG. 4 block 400, red candidate regions (R_{RR}) are received from block 200 and features are extracted for each region in block 401. Next, the red candidate regions are classified as red lesion regions (R_{RL}) and non-lesion regions (R_{NRL}) in block 402. Non-lesion red regions R_{NRL} represent false-positives and are not considered any further. All the regions that were classified as red lesions (R_{RL}) are further classified as hemorrhage regions (R_{HA}) and micro-aneurysm regions (R_{MA}) in block 403.

DR Severity Grading

Once the regions corresponding to the retinopathy lesions are detected, and the number of hemorrhages (HA), microaneurysms (MA), hard exudates (HE) and cotton-wool spots (CWS) are computed per image, the number of lesions can used to generate a DR severity grade per image as shown in FIG. 5 and FIG. 6. One embodiment of lesion combination for DR severity grading in FIG. 5 receives the fundus image in block 501, computes the number of red lesions (i.e., the number of MA and HA) in block 400, and combined the number of red lesions only to generate a DR severity grade. FIG. 5 is an embodiment of DR severity grading as per the ETDRS scale. Another embodiment of lesion combination in FIG. 6 receives the fundus image in block 601, detects red and bright lesions in block 602 (i.e., block 602 represents the combined functionality of bright lesion detection in block 300 and red lesion detection in block 400), and generates a DR severity grade based on the number of bright and red lesions. FIG. 6 is an embodiment of DR severity grading as per the ICDRS scale. In other embodiments different metrics to combine the number of bright and red lesions may be used.

### EXAMPLES

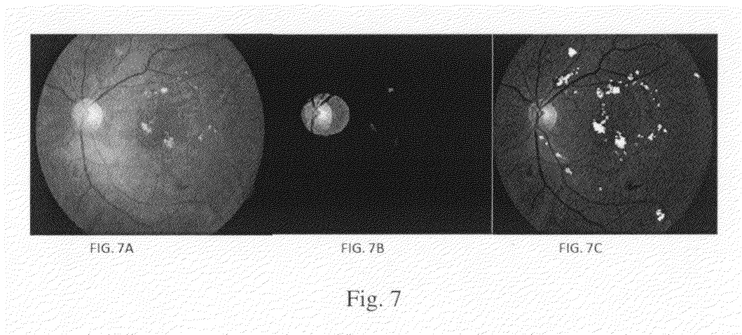

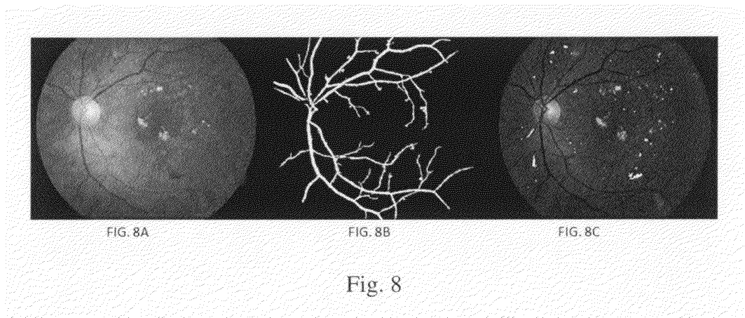

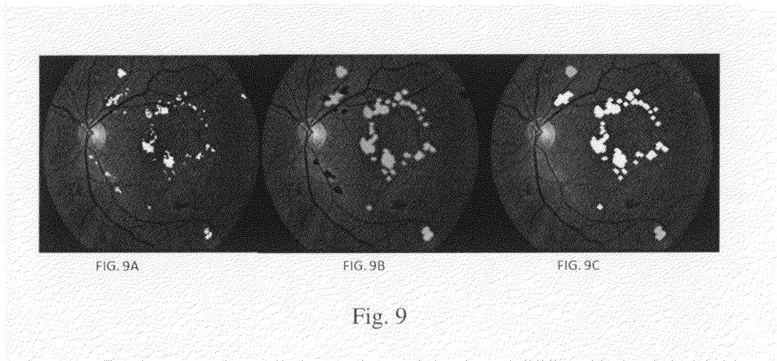

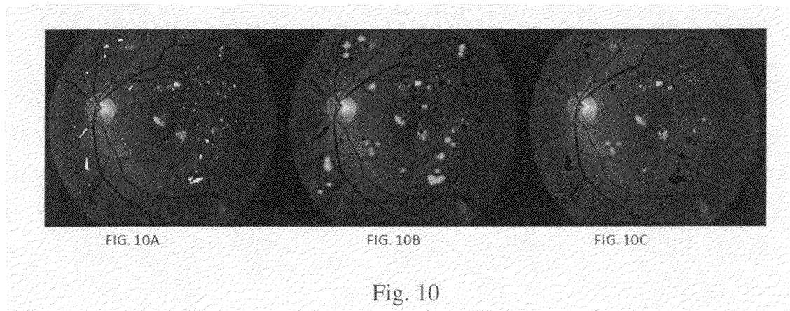

The three stages of the proposed invention are illustrated with an example in FIG. 7, FIG. 8, FIG. 9 and FIG. 10. The first stage involving extraction of bright and red candidate regions is shown in FIG. 7 and FIG. 8, respectively. FIG. 7A shows the fundus image received. FIG. 7B shows the outcome of the automated OD region detection algorithm superimposed on the original image. Next, the OD region is removed from the bright regions detected from the image, and the remaining bright candidate regions (R_{BR}) superimposed on the green plane of the fundus image are shown in FIG. 7C. The pixels marked in white in FIG. 7C represent the bright candidate regions.

FIG. 8A shows the same fundus image as in FIG. 7A. FIG. 8B shows the blood vessel regions detected and FIG. 8C shows the red candidate regions (R_{RR}) after removing the blood vessel regions from the red regions.

The second stage of the proposed invention involving classification of the bright and red candidate regions to detect retinopathy lesions is shown in FIG. 9 and FIG. 10, respectively. In FIG. 9A, the bright candidate regions from FIG. 7C (marked in white) are classified as bright lesion regions (R_{BL}), marked in gray, and non-lesion regions (R_{NBL}), marked in black regions. In FIG. 9C, the gray bright lesion regions from FIG. 9B are further classified as hard exudates (R_{HE}), marked in white, and cotton-wool spots (R_{CWS}), marked in gray.

In FIG. 10A, the red candidate regions from FIG. 8C (marked in white) are classified as red lesion regions (R_{RL}), marked in gray, and non-lesion regions (R_{NRL}), marked in black regions. In FIG. 10C, the gray red lesion region from FIG. 10B are further classified as hemorrhages (R_{HA}), marked in black, and micro-aneurysms (R_{MA}), marked in gray regions.

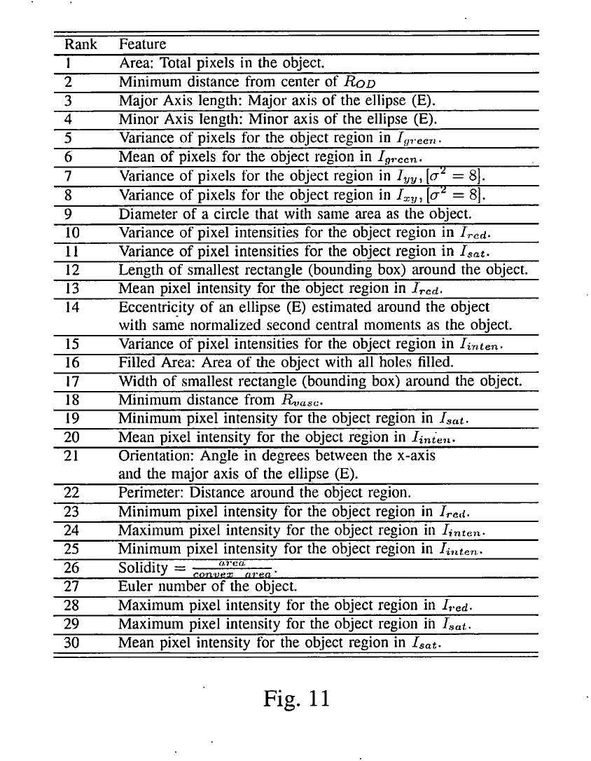

In one embodiment of the proposed invention, 30 features are chosen for the feature-based classification and detection of retinopathy lesions in the second stage of the algorithm. These 30 features were chosen by ranking 78 structural and pixel intensity-based features using AdaBoost and are shown in FIG. 11. In other embodiments, other combinations of features may be used. In various embodiments different classifiers may be used for step 1 and step 2 classification. Classifiers for step 1 need not be same for step 2. In one embodiment the Gaussian Mixture Model (GMM) classifier may be used for step 1 and step 2 for bright lesion classification. In another embodiment k-Nearest Neighbor (kNN) classifier may be used for step 1 and step 2 of red lesion classification. Examples of other classifiers include support vector machines (SVM), AdaBoost and linear discriminant analysis (LDA) classifiers. SVM classifiers may be used as linear classifiers or as non-linear (kernel) classifiers. Different embodiments may use different combinations of classifiers.

The disclosed invention is used to grade DR severity on 1200 publicly available images from the MESSIDOR dataset. Each image is segmented to detect bright and red candidate regions, followed by lesion classification and DR severity grading using the embodiment shown in FIG. 5. Finally, each image is assigned a DR severity grade 0 (indicating no DR), 1 (mild DR), 2 (moderate DR), 3 (severe DR). The performance of classifying images with grade 0 from the images with grade 1, 2, 3 are shown in FIG.

12.

Apparatus for Detecting Diabetic Retinopathy Lesions.

The methods described in this invention can be used to design an apparatus for. detecting lesions of diabetic retinopathy in fundus images. The apparatus computes the steps of the proposed methods using digital computing systems implemented using digital circuits. In one embodiment the apparatus might contain a computing system comprising a processing unit. In other embodiments, the apparatus might contain an embedded device such as a tablet computer. The embedded device may further comprise a controller that implements the methods described in the invention. The apparatus may be implemented using integrated circuits. The embedded system may contain a Field Programmable Gate Array (FPGA). The methods described in this invention can be implemented using hardware or software or combinations of both. The apparatus may be used in a telemedicine system to analyze fundus images to detect ophthalmic abnormalities. The apparatus can be integrated into a fundus camera.

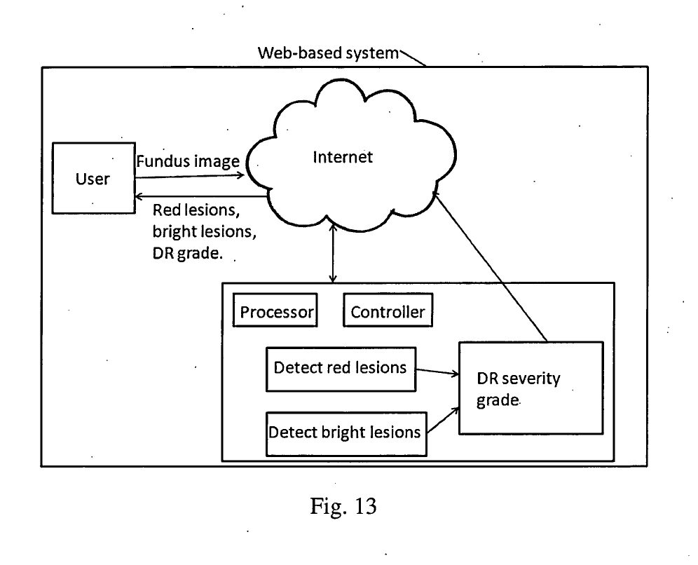

In one embodiment as shown in FIG. 13, the disclosed apparatus can be integrated into a web-development system where fundus images can be uploaded to a web system or internet. The web system server/internet then implements the methods proposed in the invention using a DR detection system that is equipped with a processor and controller, and detects the red lesions, bright lesions and DR severity grade. This DR severity grade and/or the properties and location of the red and bright lesions are then provided to the user. In another embodiment the web system may be a part of a web cloud. In a typical telemedicine system, the fundus images may be uploaded to a cloud where the proposed web-development based system apparatus can output the severity grade.

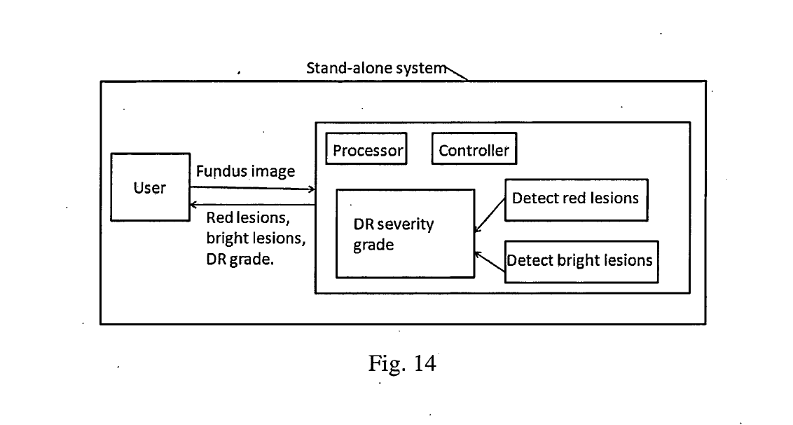

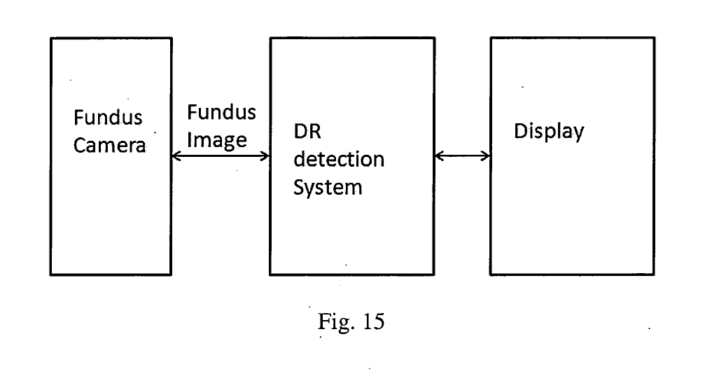

In an embodiment as shown in FIG. 14, the disclosed DR lesion detection and grading system may reside as a stand-alone system such as in a tablet computer or a cell phone or in another embedded terminal. The embedded device has a processing and controller unit that receives a fundus image from a user via the internet or via some external memory unit. The device then utilizes the disclosed invention to detect the retinopathy lesions and DR severity grade and returns them to the user. In another embodiment shown in FIG. 15, the proposed apparatus may be integrated to a fundus camera. Here, the fundus image from the camera is input to the embedded device that implements the proposed DR detection system and a suitable display is then generated.

### CONCLUSION

Specific embodiments of the present invention have been described above for fundus images with varying fields of view (FOV), illumination and abnormalities. These embodiments can be used for automated screening of DR to reduce the number of patients that need to be manually assessed, and to help prioritize follow-up treatment. It should be understood that these embodiments have been presented by way of example only, and not limitation.

It will be understood by those skilled in the relevant art that various changes in form and details of the embodiments described may be made without departing from the spirit and scope of the present invention as defined in the claims. Thus, the breadth and scope of present invention should not be limited by any of the above-described exemplary embodiments, but should be defined only in accordance with the following claims and their equivalents.

What is claimed is:

1. A method to classify bright lesions from fundus images, the method comprising:
i. extracting bright candidate regions;
ii. extracting features for these candidate regions;
iii. classifying the bright candidate regions as bright lesion candidates or non-lesions;
iv. classifying the bright lesion candidates as hard exudates or cotton-wool spots.

2. The method in claim 1 where extracting bright candidate regions further comprises segmenting bright regions from the fundus image and removing the optic disc from the bright regions.

3. The method in claim 1 used wherein the number of hard exudates and/or the number of cotton-wool spots are used to grade diabetic retinopathy.

4. The method in claim 1 implemented as part of a web cloud.

5. The method in claim 1 implemented in an embedded device.

6. A method to classify red lesions from fundus images, the method comprising:
i. extracting red candidate regions;
ii. extracting features for these candidate regions;
iii. classifying the red candidate regions as red lesion candidates or non-lesions;
iv. classifying red lesion candidates as hemorrhages or micro-aneurysms.

7. The method in claim 6 where extracting red candidate regions further comprises segmenting red regions from the fundus image and removing the blood vessel regions.

8. The method in claim 6 wherein the number of hemorrhages and/or the number of micro-aneurysms are used to grade diabetic retinopathy.

9. The method in claim 6 implemented as part of a web cloud.

10. The method in claim 6 implemented in an embedded device.

11. An apparatus for extracting red lesions from fundus images, comprising:
i. a digital circuit including a controller;
ii. extraction of red candidate regions;
iii. extraction of features for these candidate regions;
iv. classification of the red candidate regions as red lesion candidates or non-lesions;
v. classification of red lesion candidates as hemorrhages or micro-aneurysms.

12. The apparatus in claim 11 used for determining a severity grade for diabetic retinopathy.

13. The apparatus in claim 11 integrated to a fundus camera.

14. The apparatus in claim 11 used in an embedded device.

15. The apparatus in claim 11 used as a part of a web cloud where a fundus image is up-loaded to the web cloud.

16. The apparatus in claim 11 used in a telemedicine system.

17. An apparatus for extracting bright lesions from fundus images, comprising:
i. a digital circuit including a controller;
ii. extraction of bright candidate regions;
iii. extraction of features for these candidate regions;
iv. classification of the bright candidate regions as bright lesion candidates or non-lesions;
v. classification of bright lesion candidates as hard exudates or cotton-wool spots.

18. The apparatus in claim 17 used for determining a severity grade for diabetic retinopathy.

19. The apparatus in claim 17 integrated to a fundus camera.

20. The apparatus in claim 17 used in an embedded device.

21. The apparatus in claim 17 used as a part of a web cloud where a fundus image is up-loaded to the web cloud.

22. The apparatus in claim 17 used in a telemedicine system.
